(19) United States
(12) Patent Application Publication
(10) Pub. No.: US 2024/0408261 A1
(43) Pub. Date: Dec. 12, 2024

(54) AROMATHERAPY HUMIDIFIER WITH AUTOMATIC ADDITION OF ESSENTIAL OILS

(71) Applicant: Zhiting Li, Shenzhen (CN)

(72) Inventor: Zhiting Li, Shenzhen (CN)

(21) Appl. No.: 18/206,632

(22) Filed: Jun. 7, 2023

(51) Int. Cl. A61L 9/14

(52) U.S. Cl. CPC A61L 9/14; A61L 2209/134

(57) ABSTRACT

An aromatherapy humidifier with automatic addition of essential oils, comprising a device host, wherein the device host comprises: a main water tank; the main water tank is provided with a main water storage cavity, and the main water storage cavity is provided with an essential oil storage box; an auxiliary water tank; the auxiliary water tank is provided with an auxiliary water storage cavity, and the main water storage cavity infuses water into the auxiliary water tank through a water guide device; the essential oil is atomized into the water and water mist in the auxiliary water tank through the essential oil atomizer, and water and essential oils are sprayed out in an atomized form through the water atomizer.

### 1. TECHNICAL FIELD

The invention relates to an aromatherapy humidifier, in particular to an aromatherapy humidifier with automatic addition of essential oils.

### 2. BACKGROUND ART

Aromatherapy humidifiers are widely used in our lives. They atomize water through ultrasonic waves to moisten the living space, prevent various physical discomforts caused by dry weather; adding essential oils can improve the fragrance of the air, which is used as aromatherapy. Therefore, household aromatherapy humidifiers have become an indispensable household appliance in addition to TVs and air conditioners. At present, for the existing aromatherapy humidifiers, essential oils are added manually, and there is no regular and quantitative addition of essential oils, and the aromatherapy effect is not constant.

### 3. SUMMARY OF THE INVENTION

In order to solve the above problems, the invention provides an aromatherapy humidifier with automatic addition of essential oils. Through the setting of the main and auxiliary water tanks, the main water tank continuously supplies water to the auxiliary water tank, and then uses the main water tank to infuse the auxiliary water tank with essential oil, so as to realize fixed-point and quantitative essential oil supply, with a high degree of automation, which can effectively solve the deficiencies in the prior art.

The invention is achieved through the following technical solutions: an aromatherapy humidifier with automatic addition of essential oils, comprising a device host, wherein the device host comprises:
a main water tank; the main water tank is provided with a main water storage cavity, and the main water storage cavity is provided with an essential oil storage box; an essential oil storage cavity is used for storing essential oils; the essential oil storage box is provided with an essential oil atomizer output downward;
an auxiliary water tank; the auxiliary water tank is provided with an auxiliary water storage cavity, and the main water storage cavity infuses water into the auxiliary water tank through a water guide device; the atomization output end of the essential oil atomizer is communicated with the auxiliary water storage cavity; the bottom surface of the auxiliary water storage cavity is provided with a water atomizer, and water and essential oils are sprayed out in an atomized form through the water atomizer and the essential oil atomizer.

Preferably, the device host further comprises a base and an inner container; the inner container is installed on the upper end of the base, and the main water tank is installed on the upper end of the inner container; the auxiliary water storage cavity is formed on the inner container avoiding the area of the main water tank.

Preferably, if further comprises a casing, and the casing is installed outside the main water tank and the auxiliary water tank; the auxiliary water tank is provided with a first mist outlet, and the casing corresponding to the first mist outlet is provided with a second mist outlet.

Preferably, the side of the main water tank on the inner container is further provided with a support; the support is installed with a luminous decoration, and the luminous decoration is located at the position of the mist outlet.

Preferably, the base is provided with an installation cavity, and a main control board, a power supply, and a radiator are installed in the installation cavity; the radiator is controlled by the main control board, and the luminous decoration is connected to the main control board and controlled by the main control board; the main control board is provided with a plurality of control buttons on the base, and the essential oil atomizer and the water atomizer are all controlled to be opened and closed by the main control board.

Preferably, the top of the main water tank is provided with an opening and closing cover plate; the opening and closing cover plate seals the top opening of the main water storage cavity, and one side of the opening and closing cover plate is provided with a finger buckle for opening the opening and closing cover plate.

Preferably, the casing is provided with a plurality of observation windows, and the main water tank is a transparent main water tank.

The beneficial effects of the invention are: the invention is provided with a main water tank, through which the main water tank continuously supplies water to the auxiliary water tank, and the auxiliary water tank can realize continuous water atomization; at the same time, an essential oil storage box is provided in the main water tank, and the essential oils are stored in the essential oil storage box; by continuously conveying the essential oils downwards, the essential oils and water are simultaneously sprayed in an atomized form, and the quantitative and timing essential oil filling method can be achieved, with a high degree of automation and a better experience.

### 4. BRIEF DESCRIPTION OF ACCOMPANY DRAWINGS

In order to explain the embodiments of the invention or the technical solutions in the prior art more clearly, the drawings that need to be used in the description of the embodiments or the prior art will be introduced hereinafter. Obviously, the drawings in the following description are only some embodiments of the invention. For those of ordinary skill in the art, other drawings may be obtained from these drawings without creative efforts.

FIG. 1 is a schematic diagram of the overall structure according to the invention;

FIG. 2 is a schematic diagram according to the invention after removing the casing;

FIG. 3 is a schematic diagram according to the invention after removing the auxiliary water tank and the casing;

FIG. 4 is a schematic diagram according to the invention after removing the main water tank and the auxiliary water tank;

FIG. 5 is a schematic diagram of the internal structure of the base according to the invention.

### DESCRIPTION OF REFERENCE SIGNS

1 refers to the observation window; 2 refers to the casing; 3 refers to the opening and closing cover plate; 4 refers to the finger buckle; 5 refers to the first mist outlet; 6 refers to the base; 7 refers to the control button; 8 refers to the main water tank; 9 refers to the main water storage cavity; 10 refers to the essential oil storage box; 11 refers to the essential oil storage cavity; 12 refers to the essential oil atomizer; 13 refers to the second mist outlet; 14 refers to the auxiliary water tank; 15 refers to the luminous decoration; 16 refers to the support; 17 refers to the inner container; 18 refers to the auxiliary water storage cavity; 19 refers to the water guide device; 20 refers to the water atomizer; 21 refers to the radiator; 22 refers to the main control board; 61 refers to the installation cavity.

### 5. SPECIFIC EMBODIMENT OF THE INVENTION

All the features disclosed in the specification, or all disclosed methods or steps in the process, except for mutually exclusive features and/or steps, can be combined in any manner.

Any feature disclosed in the specification (including any appended claims, abstract and drawings), unless specifically stated, can be replaced by other equivalent or equivalent alternative features. That is, unless otherwise stated, each feature is just one example of a series of equivalent or similar features.

In the description of the invention, it needs to be understood that the orientation or positional relationship indicated by the terms “one end”, “the other end”, “outer”, “upper”, “inner”, “horizontal”, “coaxial”, “center”, “end”, “length”, “outer end”, etc. are based on the orientation or positional relationship shown in the drawings, which are only for the convenience of describing the invention and simplifying the description, rather than indicating or implying that the device or element referred to must have a specific orientation, be constructed and operated in a specific orientation, and therefore cannot be understood as a limitation of the invention.

In addition, in the description of the invention, “plurality” means at least two, such as two, three, etc., unless otherwise specifically defined.

The terms such as “upper”, “above”, “lower”, “below” and the like used in the invention to indicate a relative position in space are used to describe the relationship of one unit or feature relative to another unit or feature as shown in the drawings for the purpose of facilitating explanation. The term of the relative position in space may be intended to include different orientations of the device in use or operation other than those shown in the figures. For example, if the device in the figure is turned over, the unit described as being “lower” or “below” other units or features will be “above” the other units or features. Therefore, the exemplary term “below” can encompass both above and below orientations. A device can be oriented in other ways (rotated by 90 degrees or other orientations), and the space-related descriptors used in this article are explained accordingly.

In the invention, unless otherwise clearly defined and limited, the terms “provided”, “sleeved”, “connected”, “through”, “plug” and other terms should be interpreted broadly; for example, it can be a fixed connection, it can be a detachable connection, or integrated; it can be a mechanical connection or an electrical connection; it can be a direct connection, or an indirectly connection through an intermediate medium, and it can be an internal communication between two elements or the interaction relationship between two elements, unless specifically defined otherwise. For those of ordinary skill in the art, the specific meaning of the above terms in the invention can be understood according to specific circumstances.

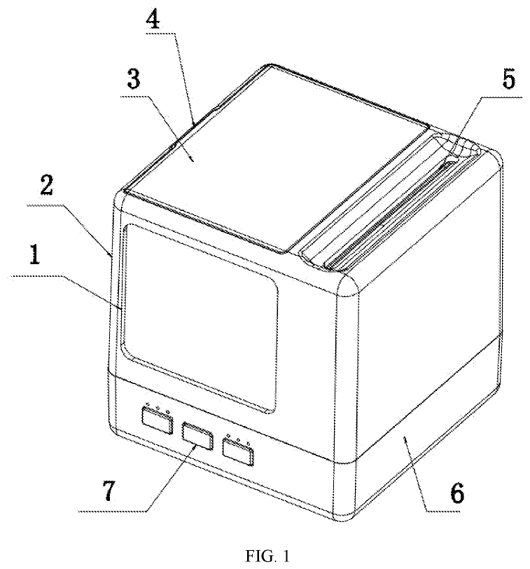

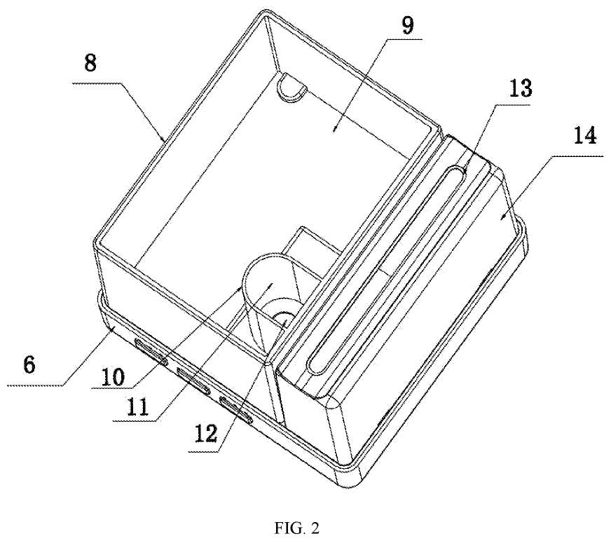

As shown in FIGS. 1 and 2, the invention provides an aromatherapy humidifier with automatic addition of essential oils, comprising a device host, wherein the device host comprises: a main water tank 8; the main water tank 8 is provided with a main water storage cavity 9, and the main water storage cavity 9 is provided with an essential oil storage box 10; an essential oil storage cavity 11 is used for storing essential oils; the essential oil storage box 10 is provided with an essential oil atomizer 12 output downward; the essential oil atomizer 12 is used to atomize the essential oil, but the atomization outlet is not on the side of the main water tank 8, so the main water tank 8 here is mainly a container for placing a large amount of water and independent essential oil;
an auxiliary water tank 14; the auxiliary water tank 14 is provided with an auxiliary water storage cavity 18, and the main water storage cavity 9 infuses water into the auxiliary water tank 14 through a water guide device 19; the atomization output end of the essential oil atomizer 12 is communicated with the auxiliary water storage cavity 18; the bottom surface of the auxiliary water storage cavity 18 is provided with a water atomizer 20, and water and essential oils are sprayed out in an atomized form through the water atomizer 20 and the essential oil atomizer 12. The auxiliary water tank 14 is mainly used to receive the water and atomized essential oil transmitted from the main water tank 8; the main water tank 8 injects essential oil and water downward, so when atomized water is generated in the auxiliary water tank 14, the atomized essential oil mixed in at this time can be discharged simultaneously with the atomized water, so as to achieve the purpose of carrying the atomized essential oil in the atomized water vapor. The essential oil storage box 10 can not only store essential oils, but also store other functional liquids, such as some Chinese herbal medicine mixtures, which can be discharged after atomization to achieve a health care purpose. In the embodiment, the water guide device can adopt a floating ball to complete the sealing and conduction by means of a mechanical seal, that is, when the water in the auxiliary water storage cavity is not enough, the floating ball will drop, and the water in the main water storage cavity will enter the auxiliary water storage cavity to replenish water, but when the water in the auxiliary water storage cavity is sufficient, the floating ball can be lifted to realize sealing; the structure is very simple, and it is also a commonly used floating ball technology.

The device host further comprises a base 6 and an inner container 17; the inner container 17 is installed on the upper end of the base 6, and the main water tank 8 is installed on the upper end of the inner container 17; the auxiliary water storage cavity 18 is formed on the inner container 17 avoiding the area of the main water tank 8. The amount of water in the auxiliary water storage cavity 18 does not need to be much; when the water amount is insufficient, water can be infused through the main water tank 8 to reduce the pressure of the auxiliary water tank 14 and ensure that the inner space of the auxiliary water tank 14 can enter the atomized essential oil. In the embodiment, by setting two water tanks, one for continuous water supply, one for continuous atomization, and there is a separate essential oil atomization component, the three are independently distributed and set, and the structural design is more reasonable.

In order to protect the main eater tank 8 and the auxiliary water tank 14, the invention further comprises a casing 2, and the casing 2 is installed outside the main water tank 8 and the auxiliary water tank 14; the auxiliary water tank 14 is provided with a first mist outlet 5, and the casing 2 corresponding to the first mist outlet is provided with a second mist outlet 13. The water and essential oil atomized by the auxiliary water tank 14 are discharged from the first mist outlet 5 and the second mist outlet 13, so that the atomized water contains atomized essential oil, which increases user experience.

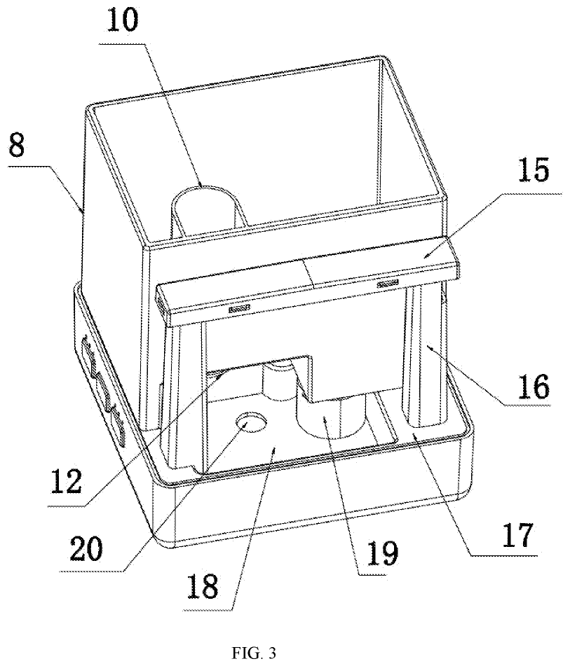

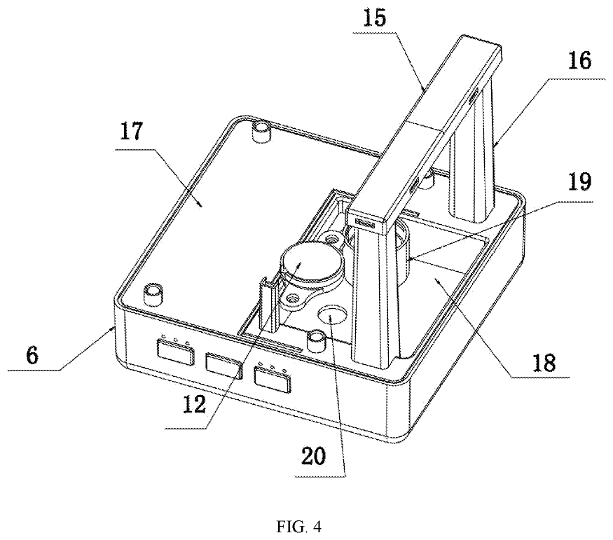

In order to increase the decorative effect, in the embodiment, the side of the main water tank 8 on the inner container 17 is further provided with a support 16, as shown in FIGS. 3 and 4; the support 16 is installed with a luminous decoration 15, and the luminous decoration 15 is located at the position of the mist outlet. By setting the luminous decoration 15 at the position of the mist outlet, the water mist can be colored, so that the atomization effect is better and more beautiful. Certainly the auxiliary water tank 14 also can adopt translucent material, which can make whole auxiliary water tank 14 have light transmittance; specifically, the luminous decoration 15 in the embodiment comprises a waterproof casing, wherein a lamp board is provided in the waterproof casing, waterproof lamp beads are arranged on the lamp board, and a light-transmitting sealing area is provided on the waterproof casing; in this way, the waterproof lamp beads correspond to the sealed area where the light is projected, and can transmit the light to achieve the effect of decoration. The assembly surface of the waterproof casing can be provided with a sealing ring to increase the sealing performance, and the whole decoration is supported and fixed by the support 16, so that the position of the decoration can be selected according to actual needs.

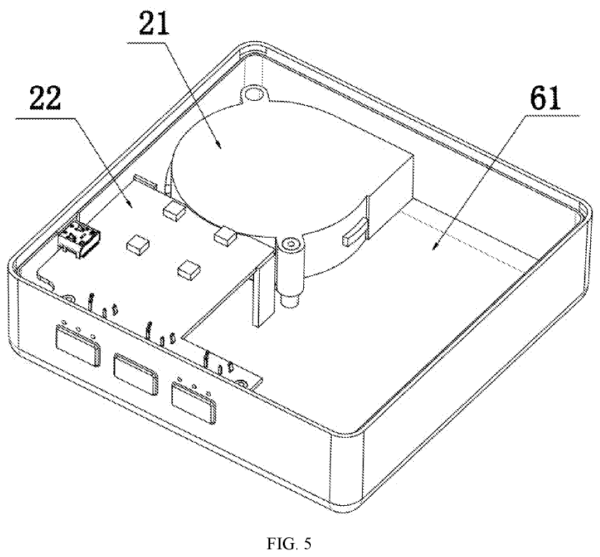

As shown in FIG. 5, the base 6 is provided with an installation cavity 61, and a main control board 22, a power supply, and a radiator 21 are installed in the installation cavity 61; the radiator 21 is controlled by the main control board 22, and the luminous decoration 15 is connected to the main control board 22 and controlled by the main control board 22; the main control board 22 is provided with a plurality of control buttons 7 on the base 6, and the essential oil atomizer 12 and the water atomizer 20 are all controlled to be opened and closed by the main control board 22. The radiator 21 can be a fan radiator 21, and heat dissipation holes are provided in the corresponding area on the base 6, so as to achieve the purpose of heat dissipation. The radiator 21 is used to reduce the heat in the peripheral area of the main control board 22, making it more stable in use and maintaining long-term use;

The control button 7 can be a switch button, a mode selection button, a light source control member, etc. The main control board 22 controls the working hours of the essential oil atomizer 12 and the water atomizer 20. The user can use the mode selection button to select a mode, and can also perform timing work, that is, after setting the opening time, the user can turn on the essential oil atomizer 12 and water atomizer 20 at the right time, and then turn off after continuing to work for a period of time. In this way, fixed-point and quantitative water mist and essential oil infusion can be controlled by the main control board 22, with a high degree of automation. This setting method is like setting a timer alarm clock; after the setting is completed, it can work according to the set time and working hours.

In order to facilitate the filling of water and essential oils, in the embodiment, the top of the main water tank 8 is provided with an opening and closing cover plate 3; the opening and closing cover plate 3 seals the top opening of the main water storage cavity 9, and one side of the opening and closing cover plate 3 is provided with a finger buckle 4 for opening the opening and closing cover plate 3. It is convenient to operate the opening and closing cover plate 3 through the finger buckle 4, and the water level scale line can be set on the main water tank 8, so that it is convenient to observe the water capacity, and it is also convenient to know the amount of essential oil. Therefore, the main water tank 8 and the essential oil storage box 10 can all be made of transparent material for easy observation.

Due to the external setting of the casing 2, which may affect the observation, in the embodiment, the casing 2 is provided with a plurality of observation windows 1, and the main water tank 8 is a transparent main water tank 8; the purpose of observation can be achieved through multiple observation windows 1.

The above are only specific embodiments of the invention, but the protection scope of the invention is not limited thereto. Any modifications or substitutions creative efforts shall all fall within protection scope of the invention. Therefore, the protection scope of the invention should be subject to the protection scope defined by the claims.

1. An aromatherapy humidifier with automatic addition of essential oils, comprising a device host, wherein the device host comprises:
a main water tank; the main water tank is provided with a main water storage cavity, and the main water storage cavity is provided with an essential oil storage box; an essential oil storage cavity is used for storing essential oils; the essential oil storage box is provided with an essential oil atomizer output downward;
an auxiliary water tank; the auxiliary water tank is provided with an auxiliary water storage cavity, and the main water storage cavity infuses water into the auxiliary water tank through a water guide device; the atomization output end of the essential oil atomizer is communicated with the auxiliary water storage cavity; the bottom surface of the auxiliary water storage cavity is provided with a water atomizer, and water and essential oils are sprayed out in an atomized form through the water atomizer and the essential oil atomizer.

2. The aromatherapy humidifier with automatic addition of essential oils of claim 1, wherein the device host further comprises a base and an inner container; the inner container is installed on the upper end of the base, and the main water tank is installed on the upper end of the inner container; the auxiliary water storage cavity is formed on the inner container avoiding the area of the main water tank.

3. The aromatherapy humidifier with automatic addition of essential oils of claim 2, wherein if further comprises a casing, and the casing is installed outside the main water tank and the auxiliary water tank; the auxiliary water tank is provided with a first mist outlet, and the casing corresponding to the first mist outlet is provided with a second mist outlet.

4. The aromatherapy humidifier with automatic addition of essential oils of claim 3, wherein the side of the main water tank on the inner container is further provided with a support; the support is installed with a luminous decoration, and the luminous decoration is located at the position of the mist outlet.

5. The aromatherapy humidifier with automatic addition of essential oils of claim 2, wherein the base is provided with an installation cavity, and a main control board, a power supply, and a radiator are installed in the installation cavity; the radiator is controlled by the main control board, and the luminous decoration is connected to the main control board and controlled by the main control board; the main control board is provided with a plurality of control buttons on the base, and the essential oil atomizer and the water atomizer are all controlled to be opened and closed by the main control board.

6. The aromatherapy humidifier with automatic addition of essential oils of claim 2, wherein the top of the main water tank is provided with an opening and closing cover plate; the opening and closing cover plate seals the top opening of the main water storage cavity, and one side of the opening and closing cover plate is provided with a finger buckle for opening the opening and closing cover plate.

7. The aromatherapy humidifier with automatic addition of essential oils of claim 2, wherein the casing is provided with a plurality of observation windows, and the main water tank is a transparent main water tank.
